(19) United States
(12) Patent Application Publication
(10) Pub. No.: US 2019/0110674 A1
(43) Pub. Date: Apr. 18, 2019

(54) SURGICAL ACCESS DEVICE INCORPORATING SCOPE CLEANER

(71) Applicants: Douglas P. Allen, Los Angeles, CA (US); Robert M. Evans, Hawthorne, CA (US)

(72) Inventors: Douglas P. Allen, Los Angeles, CA (US); Robert M. Evans, Hawthorne, CA (US)

(21) Appl. No.: 15/967,357

(22) Filed: Apr. 30, 2018

Related U.S. Application Data
(60) Provisional application No. 62/574,121, filed on Oct. 18, 2017.

(51) Int. Cl. A61B 1/12; A61B 1/00; A61B 1/313

(52) U.S. Cl. CPC A61B 1/126; A61B 1/00135; A61B 17/3423; A61B 1/127; A61B 1/3132

(57) ABSTRACT

A surgical access device is configured to provide an access path via an elongated cannula so that a medical scope can be advanced into a surgical field within a patient. A housing of the surgical access device encloses a wipe assembly configured to clean a lens of the medical scope. An access passage having an axis can be defined within the housing, and the wipe assembly can be spaced from the access passage so that the medical scope is angled off of the access passage axis in order for the lens to engage the wipe assembly. Portions of the housing can define fulcrum surfaces about which the medical scope can be rotated in order to align the lens with the wipe assembly.

### CROSS REFERENCE TO RELATED APPLICATION

This application claims the benefit of the filing date of U.S. Provisional Patent Application No. 62/574,121, filed on Oct. 18, 2017, the disclosure of which is incorporated by reference herein.

### BACKGROUND

The present disclosure relates to the field of surgical access devices that include structure for warming, cleaning, and/or defogging a medical scope such as a laparoscope.

Medical scopes, such as laparoscopes and endoscopes, are commonly used in medical procedures, particularly in minimally-invasive procedures. Such medical scopes typically have a distally-placed lens combined with a proximally-placed ocular that provides visualization within a body cavity. During laparoscopic surgery, one or more small incisions are formed in the patient's abdomen and a trocar is inserted through each incision to form a pathway that provides access to the abdominal cavity. Often, a laparoscope is inserted through one of the trocars to allow a surgeon to view the operative field.

While within the body cavity, the lens of the laparoscope can become fouled by contact with body fluids or solid debris, or can become fogged, resulting in loss of vision. In order to restore vision, the clinician may remove the laparoscope from the body cavity, clean it, and reinsert it through the trocar. This causes substantial delay, and also increases the risk of contamination. Although there are some devices that purport to clean laparoscope lenses without removing the laparoscope from the patient, such devices face challenges with effectiveness, risk, reliability, ease of use, and risk to the patient if the device fails.

### SUMMARY

There is a need in the art for a surgical access system that incorporates a medical scope cleaning device that can be used to clean a medical scope without removing the medical scope from its associated trocar, is effective for multiple types and configurations of medical scopes, provides reliable and effective cleaning, and is easy to use. There is a further need for such a surgical access system that can both warm and clean a medical scope.

In accordance with one embodiment, the present specification provides a method of cleaning a medical scope lens. The method comprises moving the medical scope along a passage defined within a surgical access device so that a lens of the medical scope is positioned proximal of a wipe surface of a wipe assembly disposed within the surgical access device. The method further comprises rotating the medical scope to an off-axis orientation in which an axis of the medical scope crosses an axis of the passage and the lens is spaced from the passage, moving the medical scope so that the lens is engaged with the wipe surface while the medical scope axis is in the off-axis configuration and moving the medical scope so that the lens is wiped across the wipe surface.

Another embodiment additionally comprises engaging the medical scope with a proximal fulcrum surface, and in such an embodiment rotating the medical scope to the off-axis orientation comprises rotating the medical scope about the proximal fulcrum surface.

Some such embodiments comprise rotating the medical scope about the proximal fulcrum surface until the medical scope engages a distal fulcrum surface, and additional embodiments additionally comprise moving the medical scope back into alignment with the passage after the lens is wiped across the wipe surface, and advancing the medical scope along the passage and into a surgical field.

Some such embodiments additionally comprise rotating the medical scope about the proximal fulcrum surface and out of engagement with the distal fulcrum surface so as to wipe the lens across the wipe surface along a back-and-forth wipe path.

Additional embodiments additionally comprise rotating the medical scope about the medical scope axis while the medical scope is engaged with both of the proximal and distal fulcrum surfaces so as to wipe the lens on the wipe surface along a twisting wipe path.

In further embodiments, the proximal and distal fulcrum surfaces are circumferential, and such embodiments may additionally comprise sliding the medical scope about the circumference of both the proximal and distal fulcrum surfaces so as to wipe the lens on the wipe surface along a swirl flow path.

Still additional embodiments additionally comprise a clinician directing a computer-controlled robot to initiate a cleaning cycle and then surrendering control of the medical scope to the computer-controlled robot, wherein during the cleaning cycle the computer-controlled robot performs the steps of moving the medical scope along the so that the lens is positioned proximal of the wipe surface, rotating the medical scope to the off-axis orientation, moving the medical scope so that the lens is engaged with the wipe surface while the medical scope axis is in the off-axis configuration, and moving the medical scope so that the lens is wiped across the wipe surface.

Yet further embodiments additionally comprise identifying a visual cue within the surgical access device visible through the lens in order to determine that the lens is adjacent the wipe assembly.

In additional embodiments, the wipe assembly comprises a foam member, and such embodiments may additionally comprise entraining a cleaning fluid in the foam member, and cleaning fluid is transferred from the foam member to the lens when the lens is wiped across the wiping surface.

Some such embodiments additionally comprise warming the cleaning fluid.

Other such embodiments additionally comprise advancing an elongated tube through an inlet formed through a side wall of the surgical access device so that an opening of the elongated tube is adjacent or engaged with the wipe assembly and delivering cleaning fluid onto the wipe assembly, and some such embodiments further comprise delivering an insufflation gas through the inlet and into the surgical access device.

In accordance with another embodiment, the specification provides a surgical access device. A housing of the device is in communication with a cannula, the housing having an access opening, the cannula having a distal opening, and an elongated passage is defined from the access opening to the distal opening, the passage having a passage axis. A wipe assembly is within the housing, the wipe assembly having a wipe surface and having a central opening aligned with the passage so that the wipe surface is spaced from the passage axis. The surgical access device further includes a proximal fulcrum surface. A line from the proximal fulcrum surface to the wipe assembly crosses the passage axis.

Some embodiments additionally comprise a distal fulcrum surface defined within the housing, the proximal and distal fulcrum surfaces configured so that a distal end of a medical scope having an elongated tubular body that is simultaneously engaged with the proximal and distal fulcrum surfaces can be advanced into contact with the wipe surface.

In some such embodiments, the proximal and distal fulcrum surfaces are circular and contiguous about their circumferences, and the wipe surface extends circumferentially about the central opening. In some embodiments the wipe surface is substantially flat.

Additional embodiments additionally comprise a heating assembly within the housing, the heating assembly comprising a heat element disposed in engagement with the wipe assembly and a controller having a sensor in engagement with the wipe assembly, the controller configured to control delivery of electrical energy to the heat element.

In some embodiments, the proximal fulcrum surface is defined by the access opening.

In additional embodiments, a valve and seal are disposed between the proximal fulcrum surface and the distal fulcrum surface.

In further embodiments, the wipe assembly comprises a foam member configured to entrain a cleaning fluid, and the wipe surface is atop the foam member.

### BRIEF DESCRIPTION OF THE DRAWINGS

FIG. 1 is a perspective view of a trocar having features in accordance with an embodiment, together with a schematic representation of a laparoscope usable with the trocar;

FIG. 2 is top end view of the trocar of FIG. 1;

FIG. 3 is an exploded view of the trocar of FIG. 1;

FIG. 4 is an exploded view of a wipe cartridge assembly of the arrangement shown in FIG. 3;

FIG. 5 is a perspective view of a wipe assembly of the arrangement show in FIG. 4 and in accordance with one embodiment;

FIG. 6 is an exploded view of the wipe assembly of FIG. 5;

FIG. 7 is a top view of the wipe assembly of FIG. 5;

FIG. 8 is a cross-sectional view taken along lines 8-8 of FIG. 7;

FIG. 9 is a cross-sectional view of the wipe cartridge assembly of FIG. 4;

FIG. 10 is a close-up view of the portion identified by lines 10-10 of FIG. 9;

FIG. 11 is a cross-sectional view taken along lines 11-11 of FIG. 2;

FIG. 12 is a close up view of the trocar of FIG. 11 showing a laparoscope using the wipe assembly in accordance with one embodiment;

FIG. 13 is a close up view of the trocar of FIG. 11 showing a laparoscope using the wipe assembly in accordance with another embodiment;

FIG. 14 is a perspective view of another embodiment of a wipe assembly and showing a plurality of lens wiping paths;

FIG. 15 is a cross-sectional view taken along lines 15-15 of FIG. 2, showing the trocar attached to a source of gas and having a laparoscope inserted therein;

FIG. 16 is an exploded view of a trocar having features in accordance with another embodiment, and an embodiment of an obturator that is configured to be insertable into the trocar;

FIG. 17 shows the assembly of FIG. 16 fully assembled;

FIG. 18 is an exploded view of the trocar of FIG. 16;

FIG. 19 is a top end view of the trocar of FIG. 18;

FIG. 20 is a cross-sectional view taken along lines 20-20 of FIG. 19;

FIG. 21 is an exploded view of a wipe cartridge assembly of the arrangement shown in FIG. 18;

FIG. 22 is a perspective view of a wipe housing of the wipe cartridge assembly of FIG. 21;

FIG. 23 is a perspective view of the wipe cartridge assembly of FIG. 21;

FIG. 24 is another perspective view of the wipe cartridge assembly of FIG. 21;

FIG. 25 is a top end view of the wipe cartridge assembly of FIG. 21

FIG. 26 is a cross-sectional view taken along lines 26-26 of FIG. 25;

FIG. 27 is an exploded view of the obturator of FIG. 16; and

FIG. 28 is a perspective view of a portion of the obturator FIG. 27.

### DESCRIPTION

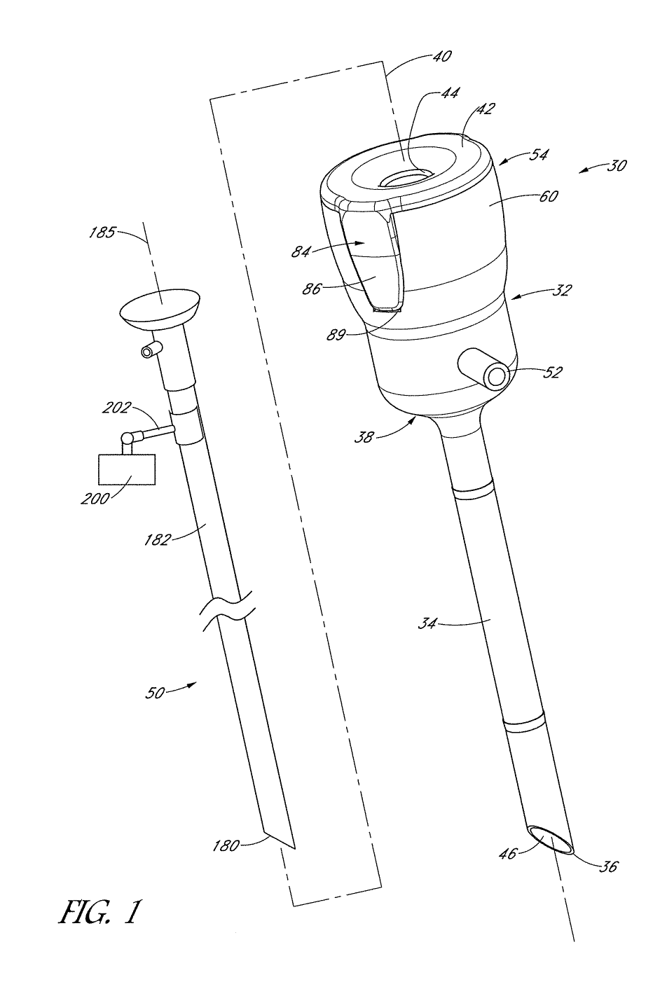

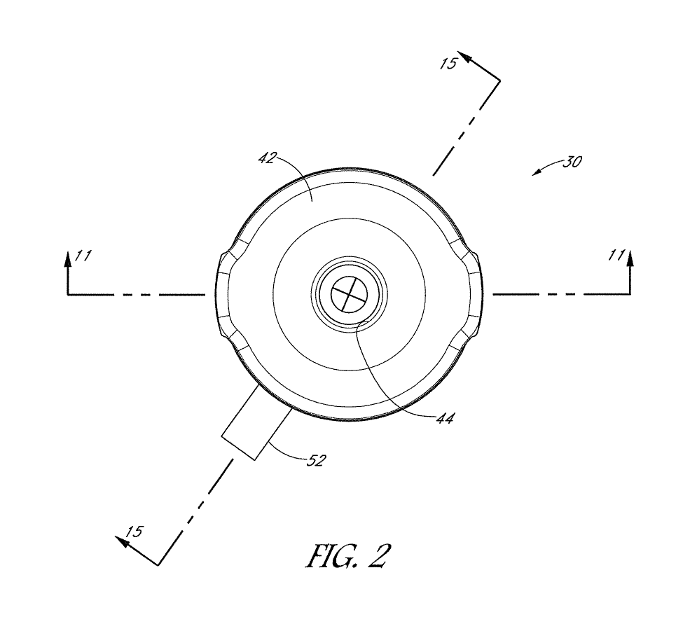

With initial reference to FIGS. 1-3, an embodiment of a surgical access device in the form of a trocar 30 is described in connection with inventive features. The illustrated trocar 30 comprises a trocar housing 32 having a relatively large diameter. An elongated cannula 34 extends from a distal end of the trocar housing 32 and terminates at a distal tip 36. The elongated cannula 34 has a much smaller diameter than the trocar housing 32. A transition portion 38 at the distal end of the trocar housing 32 reduces in diameter to match the diameter of the cannula 34. Preferably, the trocar housing 32 and cannula 34 share a longitudinal axis 40.

In the illustrated embodiment, a housing cap 42 attaches to a proximal end of the trocar housing 32 so that a space is defined within the trocar housing 32. An access opening 44 is defined through the housing cap 42, and an elongated passage is defined along the longitudinal axis 40 through the trocar 30 from the access opening 44 to a distal opening 46 at the distal tip 36 of the cannula 34. Surgical implements, such as a laparoscope 50, can be advanced through the elongated passage and into a surgical field defined within the patient's body cavity. A gas inlet 52 is provided in the trocar housing 32. An insufflation gas, such as carbon dioxide, can selectively be inserted through the gas inlet 52. Such insufflation gas can thus flow through the cannula 34 and into the patient's body cavity.

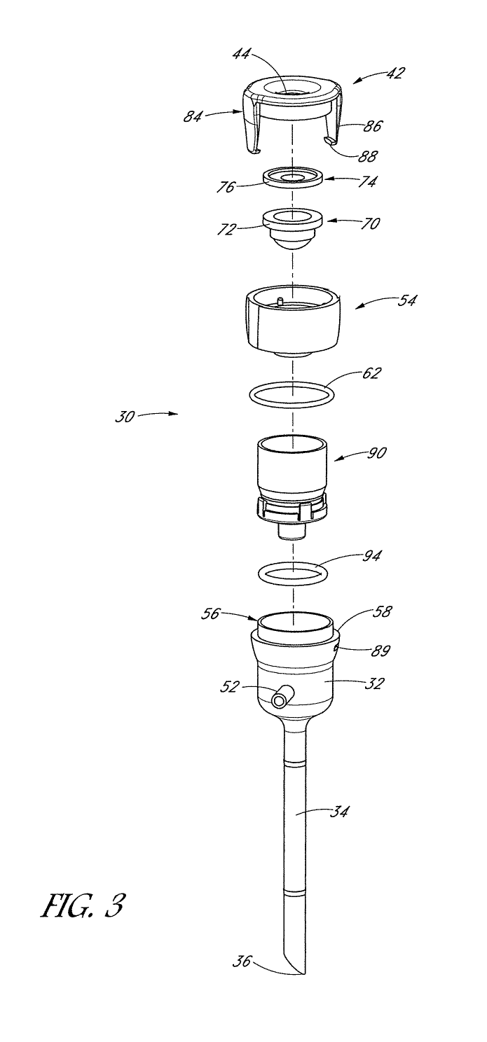

As best depicted in FIGS. 3 and 11, a seal housing 54 is sandwiched between the housing cap 42 and the trocar housing 32. A proximal receiving portion 56 is defined adjacent the proximal end of the trocar housing 32, and a seat 58 is defined at a distal end of the proximal receiving portion 56. The seal housing 54 comprises an elongated circumferential outer wall 60. A distal portion of the circumferential outer wall 60 fits over the proximal receiving portion 56 and rests upon the seat 58. A sealing O-ring 62 preferably is disposed between the proximal receiving portion 56 and the circumferential outer wall 60.

As best shown in FIGS. 3 and 11, the seal housing 54 defines a proximal seat 66 upon which a dome valve 70 can be supported. Preferably a flange 72 of the dome valve 70 engages the proximal seat 66. A seal 74 can be placed upon the proximal end of the dome valve 70, preferably with a flange 76 of the seal 74 resting atop the dome valve flange 72. A distal extension 78 of the seal housing 54 extends distally into the trocar housing and terminates in an extension opening 80. As shown, the dome valve 70 extends at least partially within the distal extension 78, which distal extension extends distally beyond the distal-most point of the dome valve 70. It is to be understood that several different specific structures for seals and valves can be employed for trocars, and various type of seals and valves are contemplated.

Continuing with reference particularly to FIGS. 3 and 11, the housing cap 42 in the illustrated embodiment comprises two clips 84. Each clip 84 includes an elongated prong 86 that terminates in an inwardly-directed projection 88. Retaining grooves 89 are formed on opposite sides of the trocar housing 32. The housing cap 42 fits over both the seal housing 54 and the proximal end of the trocar housing 32 so that the projections 88 seat themselves within the retaining grooves 89, and the housing cap 42 is thus attached to the trocar housing 32. In this configuration, the seal 74 and dome valve 70 are sandwiched securely between the housing cap 42 and the proximal seat 66 of the seal housing 54. The seal housing 54 is sandwiched securely between the housing cap 42 and the seat 58 of the trocar housing 32.

It should be understood that, in addition to or instead of the clips 84 used in the illustrated embodiment, various types of structures can be employed to attach the housing cap to the trocar housing. Additionally, in other embodiments the seal housing can be incorporated as part of the trocar housing.

In the illustrated embodiment, the extension opening 80 has a diameter greater than a diameter of the access opening 44 of the housing cap 42. As will be discussed in more detail below, inner edges of both the access opening 44 and the extension opening 80 can function as proximal and distal fulcrum surfaces 44, 80, respectively, for laparoscopes in order to aid in cleaning scope lenses.

With continued reference to FIGS. 3 and 11, a wipe cartridge assembly 90 is also enclosed within the trocar housing 32, positioned distal of the seal housing 54 and supported upon a plurality of guides 92 formed within the trocar housing 32. An absorbent ring 94, such as a cotton ring, preferably is supported between the wipe cartridge assembly 90 and the guides 92, and is positioned so as to absorb body fluids such as blood that may flow upward into the cannula 34 from the body cavity.

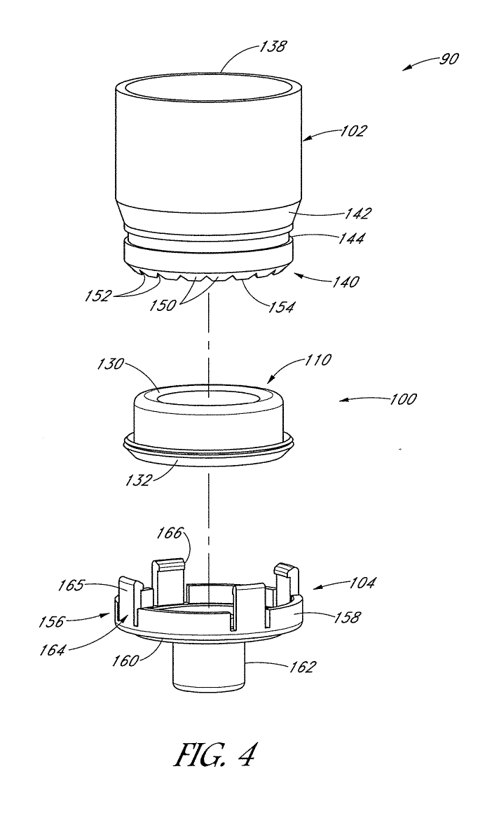

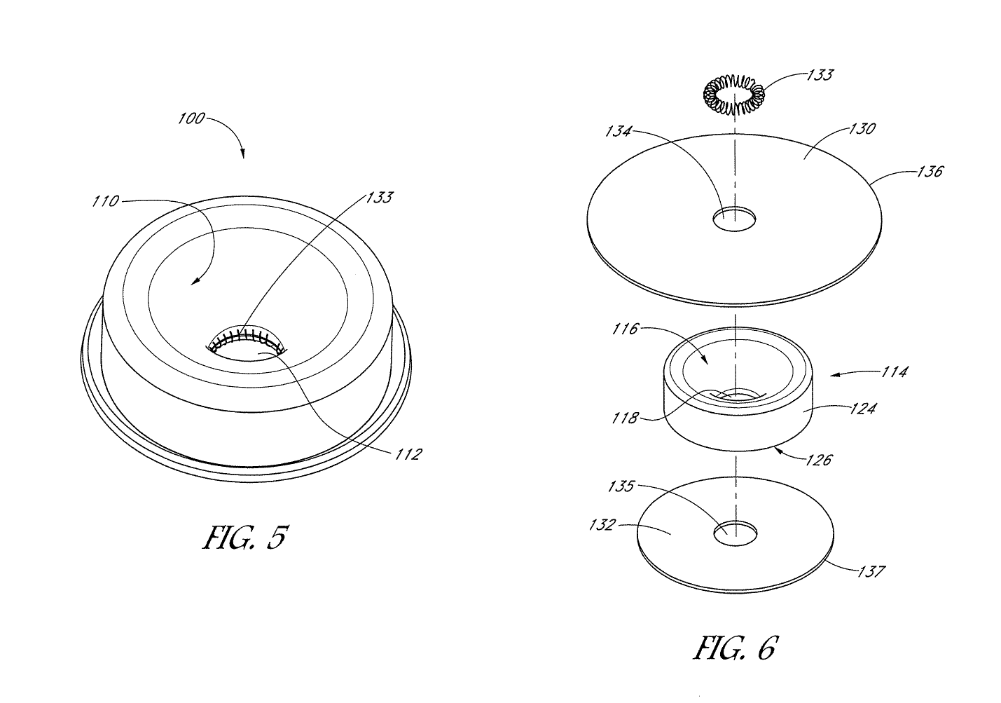

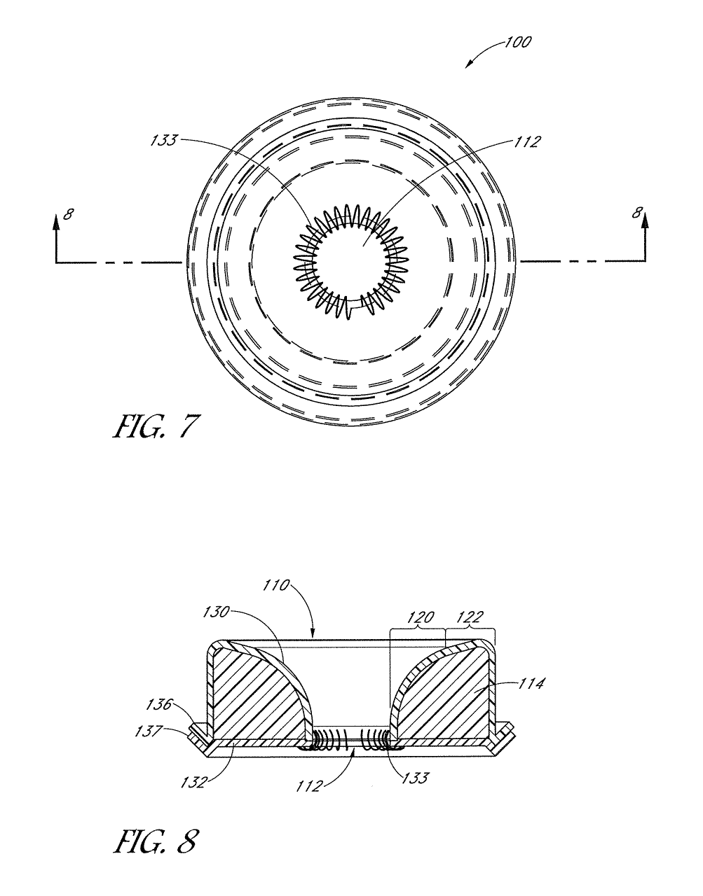

With reference next to FIGS. 4-11, the wipe cartridge assembly 90 comprises a wipe assembly 100 that is enclosed within and retained by an elongated wipe housing 102 and a wipe sleeve 104. As best shown in FIGS. 5-8, the wipe assembly 100 is generally ring-shaped, having a contoured wipe surface 110, or proximal surface, and defining a centrally-oriented opening 112 therethrough. In a preferred embodiment, the wipe assembly 100 comprises a ring-shaped foam member 114 defining a contoured proximal surface 116 and a center opening 118. The illustrated wipe assembly 100 has an arcuate zone 120 and a flat zone 122. In the arcuate zone 120, the proximal surface 116 of the foam member 114 (viewed in cross-section) follows a generally convex arcuate path moving radially outwardly from the central opening 118. The flat zone 122 is adjacent and extends radially outwardly from the arcuate zone 120. In the flat zone 122, the proximal surface 116 of the foam member 114 (again viewed in cross-section) follows a generally straight, though inclined relative to horizontal, path moving radially outwardly. An outer wall 124 of the foam member 114 preferably extends generally vertically from the proximal or contoured surface 116 to a flat bottom surface 126.

In the illustrated embodiment, the foam member 114 is captured between a wipe layer 130, or top layer, and a bottom layer 132. Preferably, both layers are, and at least the wipe layer 130 is, formed of a microfiber cloth, defining a central opening 134, 135 and an outer edge 136, 137. Preferably the wipe layer 130 and bottom layer 132 are attached to one another at their central openings 134, 135, which are aligned with the central opening 118 of the foam member 114. In the illustrated embodiment, the wipe layer 130 and bottom layer 132 are attached via a thread 133. The wipe layer 130 preferably follows and conforms to the proximal surface 116 of the foam member 114, extending over and along the contoured surface 116 and downwardly along the outer wall 124. The bottom layer 132 extends radially outwardly along the flat bottom wall 126 of the foam member 114. Preferably, the wipe layer 130 and bottom layer 132 engage one another adjacent their edges 136, 137, which extend radially outwardly from the outer wall 124 of the foam member 114. The contoured wipe surface 110 is defined on the wipe layer 130.

With additional reference again to FIGS. 4-10, the wipe assembly 100 is captured between the wipe housing 102 and the wipe sleeve 104. In the illustrated embodiment, the wipe housing 102 has an open proximal end 138 and an open distal end 140. In a transition portion 142, the wipe housing 102 decreases in diameter moving distally. A circumferential retaining groove 144 is defined on the outer surface of the wipe housing 102 distal of the transition portion 142 and spaced from the distal end 140. The distal end 140 defines a plurality of teeth 150, each of which is separated from adjacent teeth 150 by a slot 152. Preferably, the teeth 150 are tapered moving toward the distal end 140, each preferably terminating at a distal edge 154.

The wipe sleeve 104 comprises a proximal portion 156 configured to receive the distal end 140 of the wipe housing 102. The proximal portion 156 includes a proximal wall 158, which is generally circumferential and which terminates at a transverse wall 160. A tubular distal portion 162 extends distally from the transverse wall 160, which includes an opening aligned with the tubular distal portion 162. In the illustrated embodiment, the proximal portion 156 comprises a plurality of proximally-extending clips 164, each of which comprises an elongated prong 165 that terminates in an inwardly-directed projection 166. As the distal end 140 of the wipe housing 102 is advanced into the proximal portion 156 of the wipe sleeve 104, the projections 166 fit into the retaining groove 144, thus retaining the wipe housing 102 in engagement with the wipe sleeve 104.

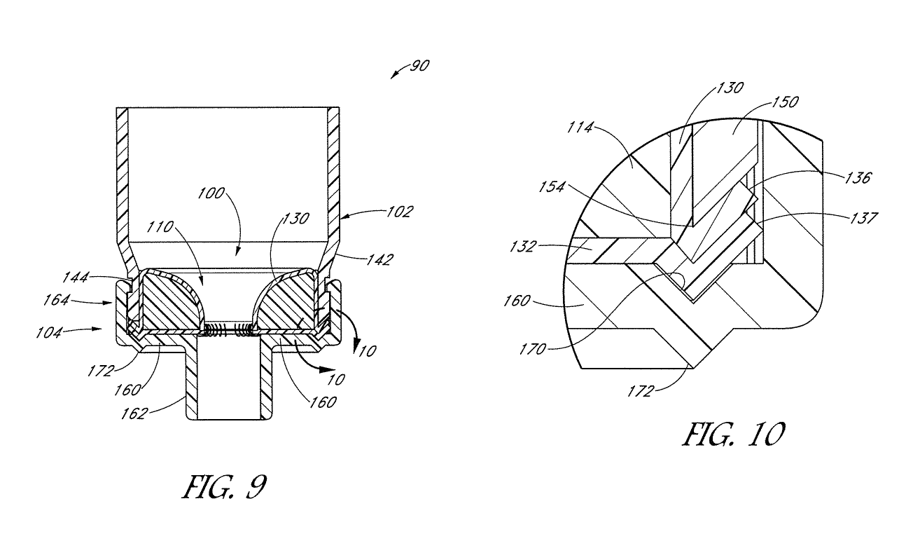

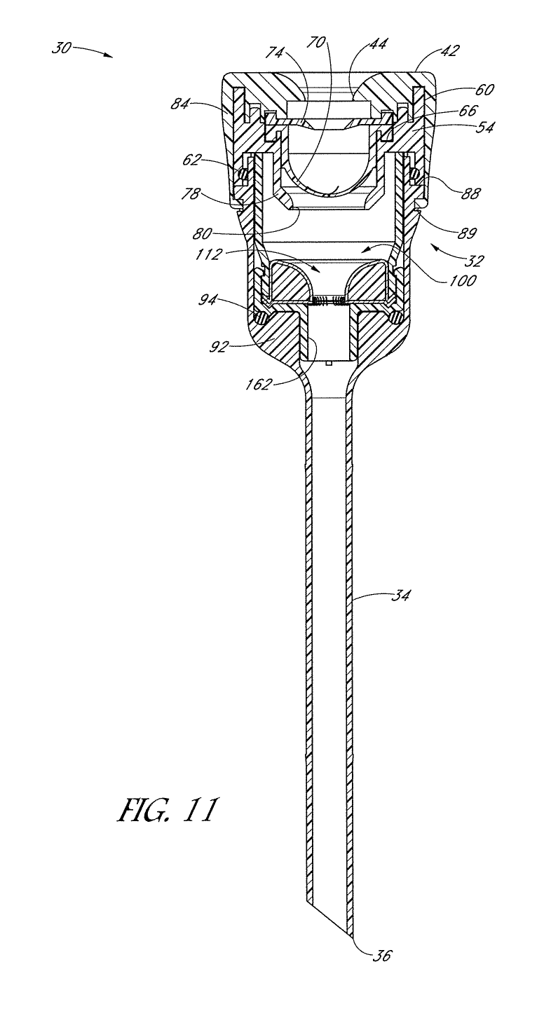

With particular reference to FIGS. 9 and 10, a circumferential groove 170, such as a V-shaped groove, is formed in the proximal surface of the transverse wall 160. Preferably the circumferential groove 170 is positioned slightly radially outwardly of the outer wall 124 of the foam member 114, and in alignment with the wipe housing teeth 150. A raised rib 172 is disposed on the distal side of the transverse wall 160 opposite the circumferential groove 170. In the illustrated embodiment, the wipe layer 130 and bottom layer 132 are aligned in engagement with one another at the circumferential groove 170, and the teeth 150 at the distal end 140 of the wipe housing 102 engage and urge the aligned wipe layer 130 and bottom layer 132 into the circumferential groove 170. When the wipe housing 102 has been advanced into the wipe sleeve 104 and is being retained in place by the clips 164, the wipe layer 130 and bottom layer 132 are sandwiched securely between the teeth 150 at the distal end 140 of the wipe housing 102 and the circumferential groove 170 in the wipe sleeve 104.

In this arrangement, not only is the foam member 114 securely held between the wipe layer 130 and bottom layer 132, but the entire wipe assembly 100 is held in position securely within the wipe cartridge assembly 90, with the wipe layer 130 drawn taut over the foam member 114. In this manner, although the wipe layer 130 is held securely in position, it can readily deform with the foam when a surgical implement such as a laparoscope lens 180 is pushed into engagement with the contoured surface. In some embodiments, the edge 154 of one or more teeth 150 is sufficiently sharp, and the teeth 150 are engaged with the wipe layer 130 with sufficient force, so that one or more of the teeth at least partially penetrates the wipe layer 130. In still additional embodiments, one or more of the teeth may fully penetrate the wipe layer 130 and at least partially penetrate the bottom layer 132.

The tubular distal portion 162 preferably is sized and positioned to align with the cannula 34 of the trocar 30. As such, and as shown in FIG. 11, the passage through the trocar 30 is defined from the access opening 44, through the seal 74 and dome valve 70, further through the extension opening 80, wipe assembly opening 112 and tubular distal portion 162 into the cannula 34 and out the distal opening 46 of the cannula at the distal tip 36.

Various materials can be used in the wipe assembly 90. For example, the wipe layer 130 preferably is made of a textile material particularly effective at wiping debris and the like from the laparoscope. Preferably the layer 130 is wettable and readily communicates fluid, such as the anti-fog/cleaning solution, from the foam member 114 onto the laparoscope 50. It is also preferred that the layer 130 be made of a material from which small fibers will not dislodge when wiping the laparoscope, as such fibers may interfere with the lens. In a preferred embodiment, the wipe layer comprises a microfiber lens cleaning cloth made of an 80%/20% polyester-polyamide blend. In other embodiments, the layer comprises a microfiber polyester-polyamide blend lens cleaning cloth made up of at least 70% polyester.

Various materials can also be used for the foam. In this specification, the term “foam” refers to a material that is flexible and will elastically recoil. Preferably, a “foam” also has advantageous wicking and liquid entrainment properties. In some preferred embodiments, the foam member comprises an open or closed cell foam having both elastic recoil and wicking ability. Most preferably, an open cell foam is employed having a density of about 1.0-1.8 lb./in.³, and more preferably about 1.2 lb./in.³, and a resiliency of about 35% to 45%, and more preferably about 35%. Examples of some acceptable foams that are currently available are known as acoustic, medical, and charcoal regular.

Various solutions may be used to clean the laparoscope and treat the lens to resist fogging. In a preferred embodiment, the anti-fog/cleaning fluid comprises 85% distilled water, 13% isopropyl alcohol and 2% surfactant. Of course, it is contemplated that other solutions may be acceptably employed as desired.

In a preferred embodiment, cleaning fluid can be added to the foam member 114 during manufacturing of the trocar 30. In another embodiment, the trocar 30 is provided partially disassembled, and cleaning fluid is added to the wipe assembly 90 during assembly of the trocar 30 prior to the procedure. In still further embodiments, a pipette or the like is inserted through the access opening 44 and into engagement with the wipe assembly to deliver cleaning fluid to fill the foam member 114 immediately prior to or in the early stages of the procedure.

It is to be understood that any of several strategies and structures can be employed to fill the wipe assembly 100 with cleaning fluid so that the cleaning fluid become entrained in the foam. For example, in another embodiment, the gas inlet port 350 (which can be equipped with an on/off valve) can be positioned immediately above the wipe assembly 100. In one such embodiment, a syringe having cleaning fluid can be secured to and/or advanced through the gas port, and the cleaning fluid can be injected via the port into the wipe assembly using, for example, an elongated tube such as a syringe.

It is also to be understood that other embodiments may employ different structure for the wipe assembly. For example, in some embodiments there will be only a wipe layer and no bottom layer. In further embodiments, instead of a wipe layer, only the foam member is provided, and the foam will operate as its own wipe layer. In still further embodiments, the wipe layer may be adhered or otherwise attached to the foam, and no portion of the wipe assembly is or need be sandwiched between the wipe housing and wipe sleeve. In yet further embodiments, the foam member can include a portion that is sandwiched between, or otherwise attached to, the wipe housing and web sleeve. In still other embodiments, the wipe assembly may be attached or secured to a wipe housing or the like using still other structure. In any case, preferably a contoured wipe assembly having a contoured wipe surface and central aperture is provided within the trocar housing.

As is known in the art, a laparoscope 50 can be inserted through a trocar 30 and into a patient's body cavity so as to provide viewing during a surgical procedure. The trocar embodiment as described herein provides this utility. However, the present trocar 30 also provides the ability to wipe the lens 180 of such a laparoscope 50, as well as to apply a cleaning solution such as anti-fog solution thereto, without removing the laparoscope 50 from the trocar 30. Further, embodiments herein can provide effective lens cleaning for laparoscopes of various configurations, such as those having a 0°, 30°, or 45° lens angle.

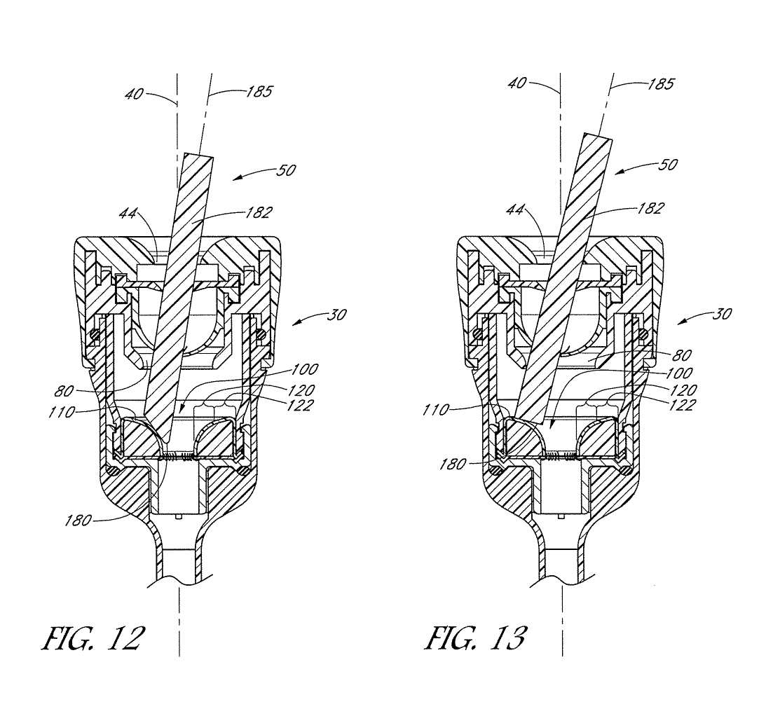

With reference next to FIG. 12, a method for cleaning the lens 180 of a laparoscope 50 having a 45° lens angle is discussed. In the illustrated embodiment, the lens is arranged at a 45° angle relative to an elongated laparoscope body 182. As shown, when the lens 180 is positioned adjacent the wipe assembly 100 wipe surface 110, the laparoscope 50 is rotated so that the laparoscope axis 185 is angled relative to the trocar axis 40 (i.e., “off axis”), the lens 180 engages the wipe surface 110 along the contoured proximal surface of the wipe assembly 100. The contoured wipe surface 110 will contact all or most of the 45°-oriented lens 180. Preferably the clinician will urge the laparoscope 50 into substantial engagement with the wipe assembly 100 so that the lens 180 engages the wipe surface sufficient so that the wipe surface 110 can effectively wipe debris and the like from the lens 180, and so that the underlying foam member 114 is deformed sufficiently to release cleaning fluid entrained therein.

Once the 45° lens is engaged with the contoured zone 120 of the wipe surface 110, the clinician moves the lens 180 back and forth radially relative to the wipe surface 110 in order to move the lens 180 over the wipe layer 110 and thus wipe debris and the like off of the lens 180. During this process, forces are applied through the wipe layer 130 to the foam 114, thus forcing cleaning fluid out of the foam 114, through the microfiber wipe layer 130, and onto the lens 180.

In the illustrated embodiment, the clinician may use the inner edge of the access opening 44 as a proximal fulcrum surface 44 to guide such off-axis movement. For example, in FIG. 12, part of the laparoscope's elongated body 182 is engaged with the access opening inner edge 44. Using the access opening inner edge as a proximal fulcrum surface 44, the clinician can then rotate the laparoscope 50 about this proximal fulcrum surface 44 (keeping the laparoscope engaged with the proximal fulcrum surface), which will in turn urge the lens 180 of the laparoscope 50 radially outward across the contoured zone 120 of the wipe surface 110 so that the wipe layer 130 of the wipe assembly 100 can wipe debris off of the lens 180. Eventually, the laparoscope body 184 will engage the inner edge of the extension opening 80, and the extension opening operates as a distal fulcrum surface 80. Engagement of the laparoscope 50 with the distal fulcrum surface 80 stops the laparoscope 50 from being rotated further about the proximal fulcrum surface 44, preventing the lens 180 from moving radially-outwardly too far, such as off of the wipe surface 110 and into contact with the inner surface of the trocar housing 32 or other structure within the trocar housing 32. The clinician can then reverse course, rotating the laparoscope 50 back into general alignment with the axis 40, further wiping the lens. The clinician can repeat this wipe motion between engaged fulcrum points to wipe the lens along a back-and-forth wipe path 190 (see also FIG. 14), while being assured both of optimal wiping of the lens and avoidance of possible damage to the lens that could perhaps result from excessive off-axis rotation. When the clinician is satisfied that the lens 180 has been sufficiently cleaned, the laparoscope 50 can be re-aligned so that its axis 185 is generally parallel to the trocar axis 40, and reinserted through the passage into the body cavity.

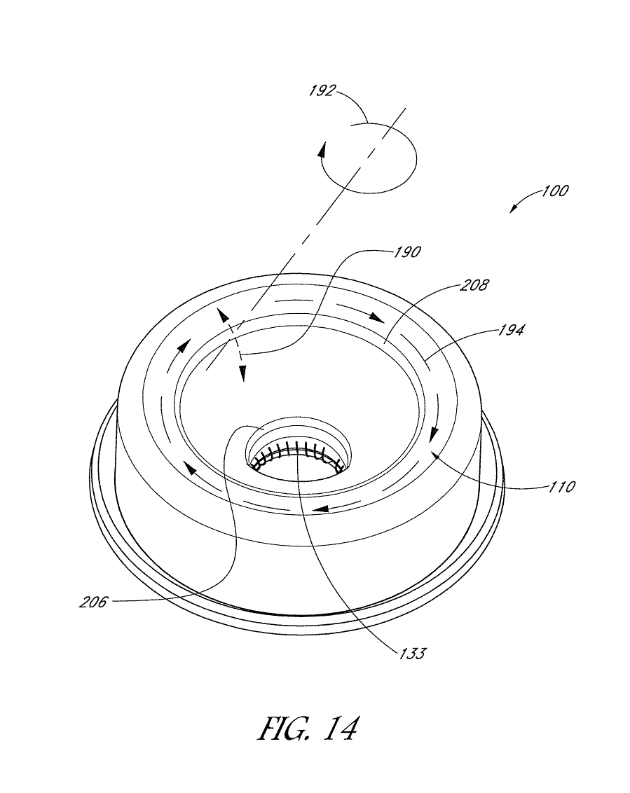

With reference next to FIG. 13, an example of cleaning a 0° laparoscope lens 180 is shown. In the illustrated embodiment, after retracting the laparoscope 50 sufficiently so that the lens 180 can access the wipe assembly 100, the clinician rotates the laparoscope off axis sufficiently so that it engages both the proximal fulcrum surface 44 and distal fulcrum surface 80. Preferably, the diameters of the access opening (which defines the proximal fulcrum surface) and extension opening (which defines the distal fulcrum surface) are selected so that, for laparoscopes within a selected range of outer diameters, the lens of the 0° laparoscope will be generally engaged with the flat zone 122 of the wipe assembly 100, assuring good wipe engagement between wipe assembly and the 0° lens. At this point, the clinician can, in one embodiment, simply twist the laparoscope 50 about its axis 185 in order to wipe the lens 180 along a twist wipe path 192 (see also FIG. 14). In another embodiment, the clinician can, while maintaining the scope body 184 in contact with both the proximal and distal fulcrum surfaces 44, 80, rotate the laparoscope 50 in a swirling motion so that the scope body 184 slides about the circumferences of the fulcrum surfaces 44, 80 and the laparoscope lens 180 slides along a circumferential swirl wipe path 194 about the wipe layer 130 (see also FIG. 14). Once the clinician is satisfied that the lens 180 is sufficiently wiped, and cleaning fluids sufficiently applied, the laparoscope 50 is again longitudinally aligned with the trocar axis 40 and advanced into the patient's body cavity.

In some embodiments, scopes having angled lenses, such as 30° and 45° lenses, can also be wiped using the swirling motion. Care preferably is taken to maintain the lens 180 in contact with the wipe surface 110 during such swirling. In some embodiments, the scope body may have an indicator visible from outside the trocar housing to indicate which side of the laparoscope has the angle lens, thus enabling the clinician to appropriately rotate the laparoscope 50 about its own axis 184 during swirling motion in order to keep the lens engaged with the wipe surface.

It is to be understood that, in the illustrated embodiment, positions of the fulcrum surfaces are selected so that a laparoscope having a diameter (or width) within a desired, or anticipated, range, will be guided so that the lens of the laparoscope is placed appropriately on the wipe assembly. In the illustrated embodiment, selection of fulcrum surface positions 44, 80 is accomplished by selecting the diameters of the access opening and extension opening, as well as the distance between these structures. In the illustrated embodiment, the extension opening 80 has a greater diameter than the access opening 44. It is to be understood that, in other embodiments, different structures may be employed for the fulcrum surfaces, and appropriate selection, spacing and sizing of such structures is made so as to appropriately guide, and preferably limit, movement of the laparoscope lens 180 during wiping/cleaning operations. Additionally, the fulcrums provide both guidance and support for a clinician manipulating a laparoscope. Thus, it is anticipated that a cleaning operation can be accomplished by a clinician using only one hand.

In the illustrated embodiment, the proximal and distal fulcrum surfaces 44, 80 are each contiguous about their entire circumference, enabling a swirling motion of the laparoscope 50 and correct positioning of the laparoscope anywhere about the circumference of the trocar axis in other embodiments, such as embodiments in which the foam member may not be ring-shaped may be disposed only one side of the trocar housing, one or more of the fulcrum surfaces may have a limited zone of operation that corresponds to a limited range of correct positioning of the laparoscope with the lens engaged with the foam member.

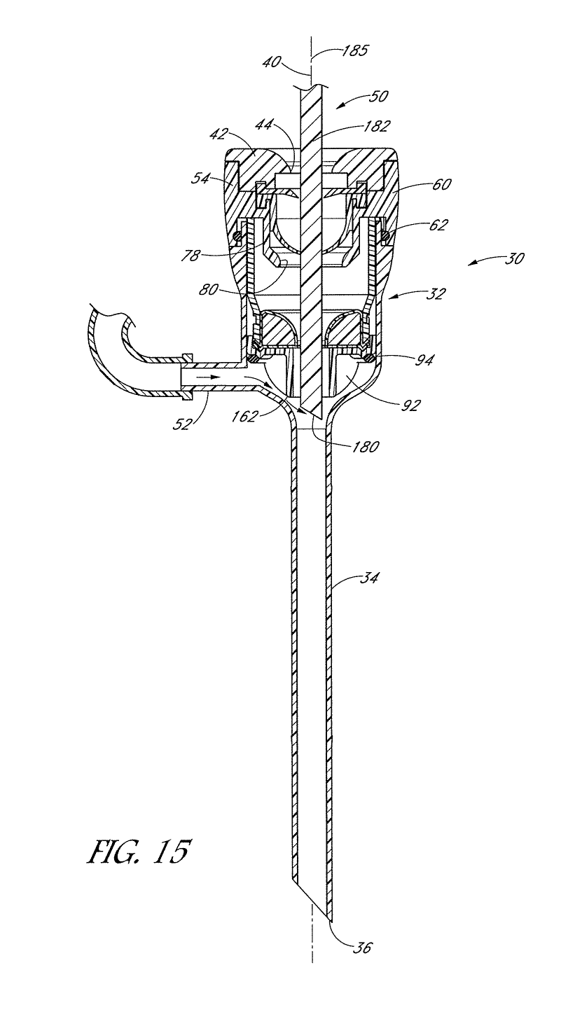

With reference next to FIG. 15, and in still another, optional, embodiment, after the laparoscope 50 has been wiped and had cleaning solution applied thereto, the laparoscope can be advanced through the wipe assembly 100 distally to a location adjacent the gas inlet 52. Insufflation gas is then injected into the trocar housing 32 and flows against the laparoscope lens 180, thus drying the lens and further enhancing visibility. The clinician can thus proceed with performing the procedure with a wiped and dried lens. In the illustrated embodiment the gas inlet 52 is positioned below, or distal, the wipe assembly. In other embodiments the gas inlet is positioned above, or proximal, the wipe assembly. In some embodiments the clinician may not wish to dry the lens as part of the cleaning operation.

It is to be understood that surgical procedures may entail several cleaning operations. And since the cleaning operation is performed within the trocar housing 32, and more preferably within a sealed portion of the trocar housing 32 distal of the seal 74 and dome valve 70, the risk of contamination is lowered, and cleaning procedures are both enhanced and more time-efficient. For example, preferably the lens is cleaned within the housing 32 sufficiently distal of the seal 74 to reduce or eliminate the likelihood that any contaminates on the seal would be transferred to the lens.

It is also be understood that the cleaning procedures are particularly advantageous for robotic surgery. For example, with additional reference to FIG. 1, a computer-controlled surgical robot 200 can have a control arm 202 that selectively engages and controls the laparoscope 50. The surgical robot 200 will be able to employ the fulcrum surfaces 44, 80 as reference points to ensure that the laparoscope 50 is properly engaged with the wipe assembly 100, sufficiently cleaned, and that the laparoscope does not mistakenly leave the wipe assembly 100, which could potentially damage the lens 180 or expose the lens to additional debris. In some embodiments, a cleaning sub-routine can be programmed into robotic procedures so that the clinician can independently control the laparoscope 50, but surrender control of the laparoscope to the robot 200 for cleaning procedures. In such an embodiment, the clinician could simply initiate the cleaning sub-routine and the robotic system will perform the sub-routine without requiring input or control of the clinician. When the cleaning sub-routine is complete, the robot 200 can emit a signal, prompting the clinician to resume control of the laparoscope 50.

In additional embodiments, various visual cues and landmarks can be used to help the clinician, or robot as the case may be, identify the correct location of the laparoscope lens 180 relative to the trocar 30, wipe assembly 100, and the like. For example, in some embodiments, the wipe sleeve 104 is formed of a colored material, preferably a brightly colored polycarbonate. As such, when the clinician is withdrawing the laparoscope, once this bright color is identified, the clinician knows that the lens 180 is approaching the wipe assembly 100. In additional embodiments, the thread 133 at the distal end of the wipe assembly distal opening 112, which attaches the wipe layer 130 to the bottom layer 132, is brightly colored using another high-contrast color, thus alerting the clinician that the lens has passed through the central opening 112 and is now immediately adjacent the wipe assembly contoured surface 130. The clinician thus will know it is now appropriate to proceed with a cleaning procedure. In still further embodiments, and with specific reference to FIG. 14, brightly-colored, high-contrast bands can be applied to the wipe layer 130. For example, a first colored band 206 can be applied close to, but spaced from, the central opening 112, signaling the clinician that, for example with a 45° laparoscope lens, it is time to begin rotating the laparoscope off axis so that the lens moves radially to wipe the lens. A second colored band 208 can signal the clinician that the lens has reached the limit of radially-outward wiping, and it is time to wipe in the other direction.

In some embodiments, the second colored band 208 can correspond to the transition between the arcuate zone 120 and flat zone 122 of the wipe surface 110. Thus, the second colored band 208 signals the user of a 45° or 30° laparoscope 50 that the end of the arcuate zone 120 has been reached and it is time to move the scope 50 in another direction. However for a clinician using a 0° scope, the clinician may wish to align the scope with or adjacent the second colored band 208 so that the clinician can be assured that the 0° lens 180 is aligned with the flat surface 122 for optimal wiping.

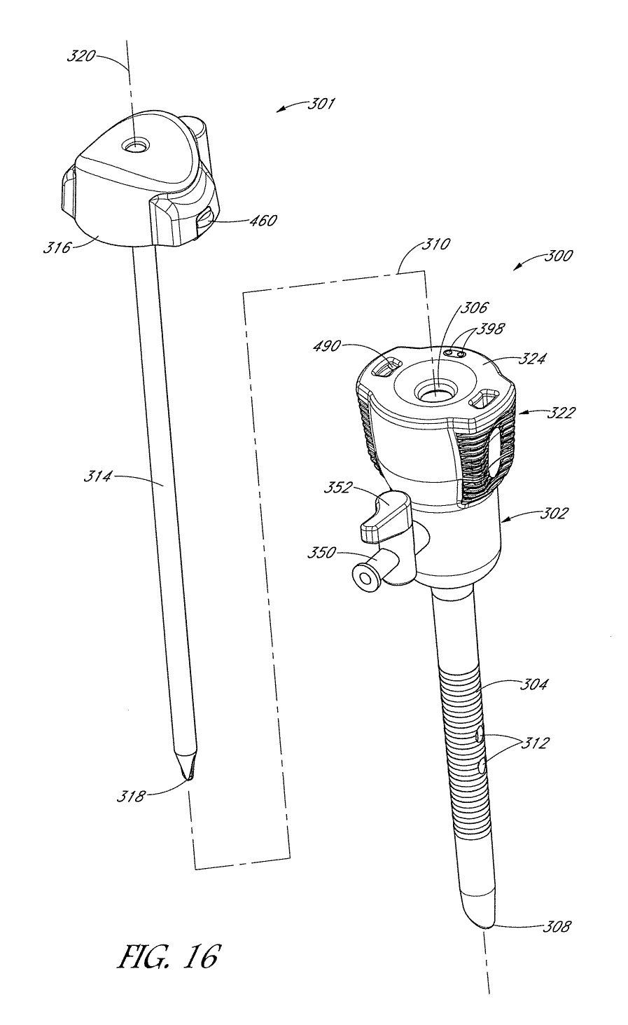

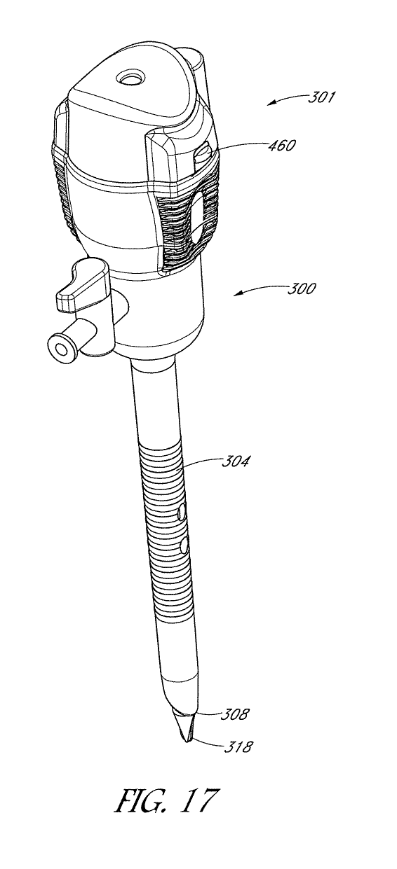

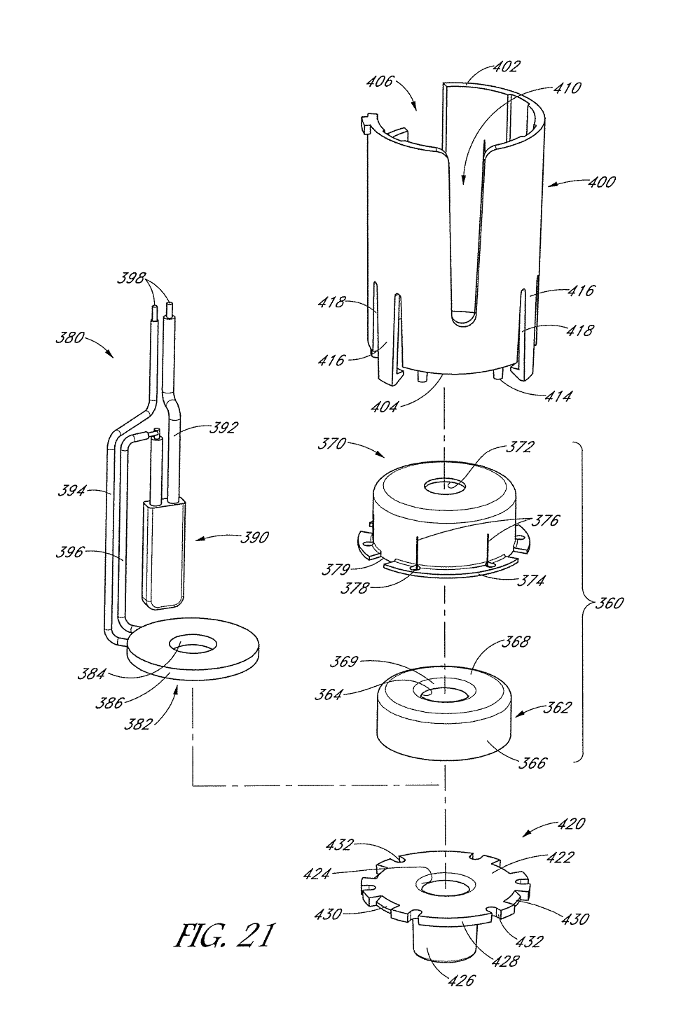

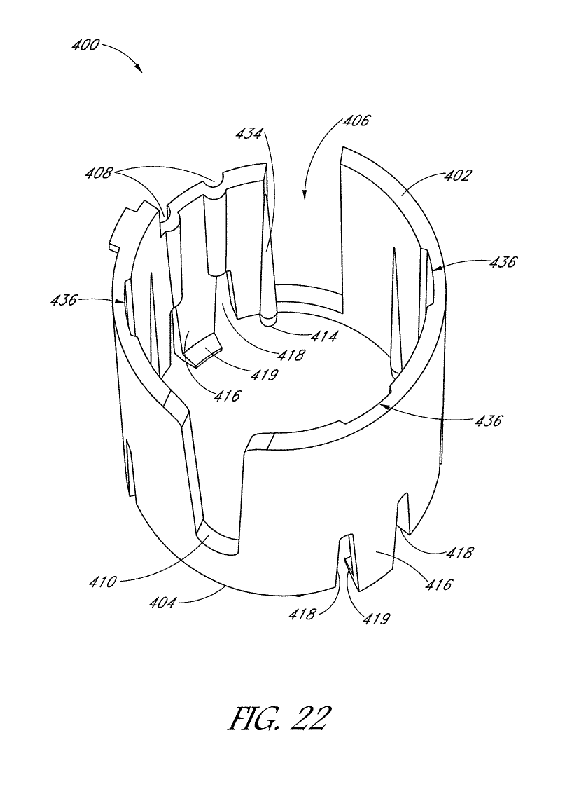

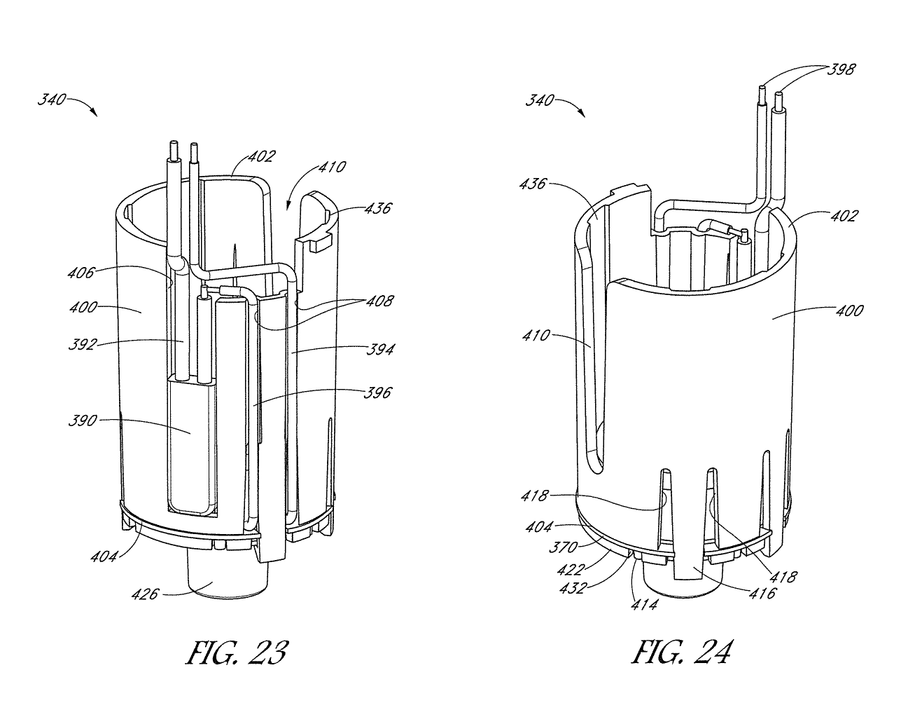

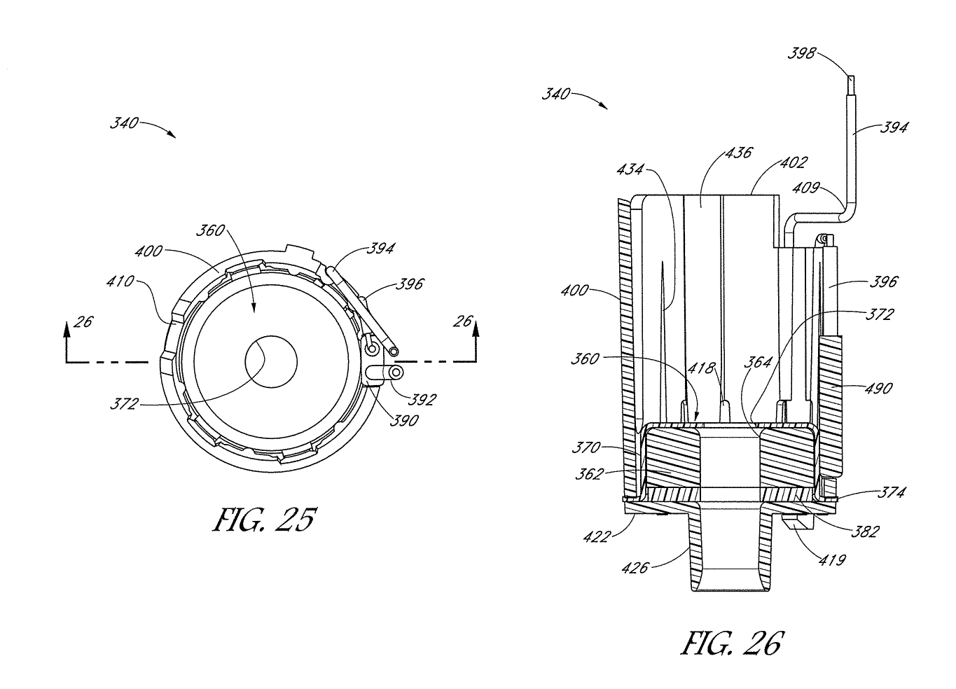

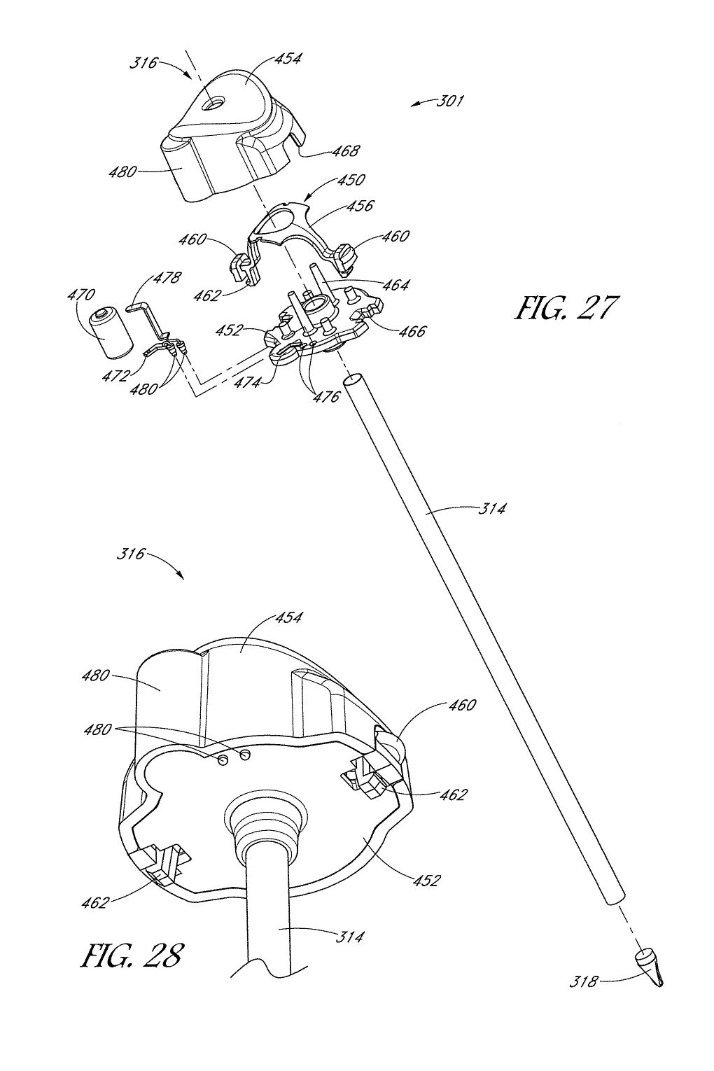

With reference next to FIGS. 16-28, another embodiment of a surgical access device is illustrated. With specific reference first to FIGS. 16 and 17, a trocar 300 is configured to receive an obturator 301 in order to initiate surgical access to a patient's abdomen. The illustrated trocar 300 includes a trocar housing 302 having an elongated cannula 304 extending distally therefrom. An elongated passage is defined through the trocar 300 from an access opening 306 at its proximal end to a distal tip 308. Preferably, the passage lies along a trocar axis 310. In the illustrated embodiment, a plurality of side openings 312 are formed through a side wall of the cannula 304. The obturator 301 includes an obturator shaft 314 having an obturator handle 316 at its proximal end and terminating at an obturator point 318 at its distal end. The shaft 314 and point 318 lie along an obturator axis 320. The obturator point 318 and shaft 314 can be extended through the trocar access opening 306 and out the distal tip 308, as depicted in FIG. 17, preparatory to using the combined device to establish abdominal access.

With reference next to FIGS. 18-20, the trocar 300 comprises a seal housing 322 sandwiched between a cap 324 and the trocar housing 302. A seat 326 of the trocar housing 302 is configured to receive the seal housing 322 resting thereon. In the illustrated embodiment, the seat 326 includes a plurality of guideposts 328 configured to engage corresponding receivers (not shown) in the seal housing 322 to ensure proper alignment. In a preferred embodiment, the seal housing 322 is press-fit into the seat 326 of the trocar housing 302 for permanent assembly. A sealing O-ring 329 preferably is disposed between the trocar housing 302 and seal housing 322.

A duckbill valve 330 has a flange 332 and is configured so that the flange 332 rests upon a seal housing seat 335. A seal 334 having a flange 336 preferably rests upon the duckbill valve 330 such that flange 336 rests upon flange 332. A plurality of seal housing guideposts 338 are configured to complementarily engage corresponding receivers (not shown) in the cap 324 so that the cap 324 is received by the seal housing 322 with the seal 334 and duckbill valve 330 sandwiched securely therebetween.

It is anticipated that other embodiments may employ different specific structures of seals and valves (such as a dome valve) as well as specific structures for housing such seals and valves. Also, in some embodiments the seal housing 322 can be releasably attached to the trocar housing 302 and the cap 324 correspondingly releasably attached to the seal housing 322. In other embodiments, one or more of such attachments can be permanent, such as via adhesive or clips.

With continued reference to FIGS. 18-20, a wipe cartridge assembly 340 is disposed within the trocar housing 302 distal of the seal housing 322. Preferably, a distal end of the wipe cartridge assembly 340 is supported by a plurality of guides 342 within the trocar housing 302. In the illustrated embodiment, an absorbent sheet 344, such as a cotton sheet, is supported within the trocar housing 302 between the wipe cartridge assembly 340 and the guides 342. The illustrated absorbent sheet 342 comprises a plurality of sheets slots 346 extending proximally from a distal opening 348 of the sheet 344. Preferably, each sheet slot 346 is aligned with and receives a corresponding guide 342.

A gas inlet 350 is formed through a side wall of the trocar housing 302 and defines a passage for insufflation gas to be delivered into the trocar 300, from which it is directed to the cannula 304 and further through a distal opening 353 into the patient's abdomen. In the illustrated embodiment, a stopcock 352 is provided to selectively interrupt supply of insufflation gas through the gas inlet 350. As will be discussed in more detail below, an elongated tool such as a syringe can be advanced through the inlet 350 in order to deliver cleaning fluid to a wipe assembly 360 within the trocar.

With specific reference next to FIGS. 21-26, the wipe cartridge assembly 340 comprises the wipe assembly 360 enclosed within a wipe housing 400. The illustrated wipe assembly 360 comprises a ring-shaped foam member 362 having a central opening 364 and an outer wall 366. The illustrated foam member 362 comprises a generally flat proximal surface 368. A tapered surface 369 can provide a transition between the opening 364 and the proximal surface 368.

A wipe layer 370 extends over and partially encloses the foam member 362. The illustrated wipe layer 370 includes a central opening 372 that aligns with the foam member opening 364 but, in the illustrated embodiment, has a smaller diameter. The wipe layer 370 extends over the proximal surface 368 and distally over the outer wall 366, terminating at an edge 374 which, in the illustrated embodiment, is turned generally radially outwardly. A wipe surface is defined by the wipe layer 370. A plurality of elongated slots 376 are formed through the wipe layer 370 extending generally radially outwardly, but stopping short of the edge 374. In the illustrated embodiment, a portion of the slots 376 adjacent the edge 374 can be deformed to form apertures 378 by posts that can extend therethrough, as will be discussed in more detail below. In the illustrated embodiment, a plurality of cutouts 379 are provided in the wipe layer 370 to help the wipe layer generally conform to the shape of the foam member 362 and provide clearance for clips 416, as will be discussed in more detail below.

It is to be understood that, in additional embodiments, the foam member and wipe layer can be made of materials as discussed in connection with other embodiments, such as those discussed in connection with FIGS. 1-15, and can incorporate contour shapes, visual cues and other properties as in embodiments discussed above. Further, the illustrated wipe assembly can be used in a manner consistent with features of any of the above-discussed embodiments.

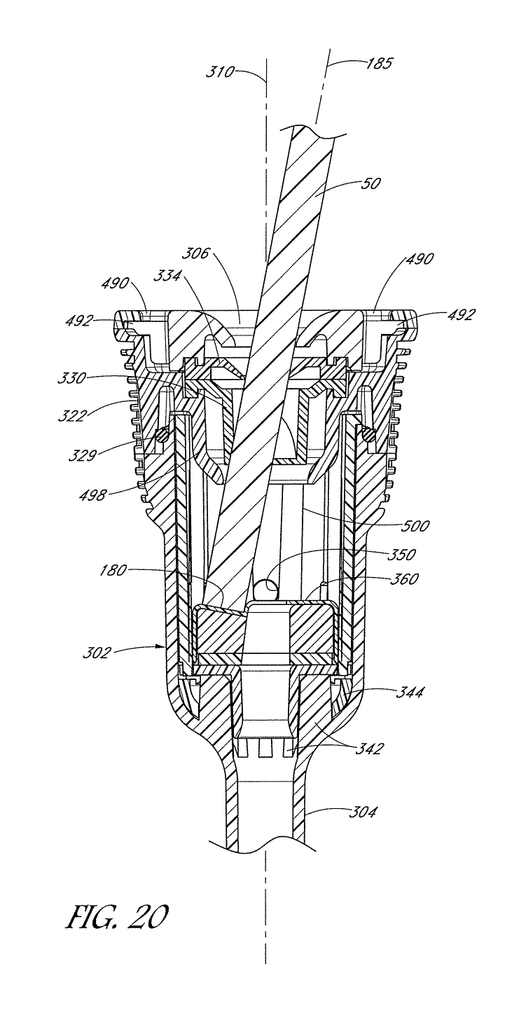

With continued reference to FIGS. 20-26, the wipe cartridge assembly 340 additionally comprises a heater assembly 380 configured to warm fluid that may be entrained within the wipe assembly 360. The illustrated heater assembly 380 comprises a disk-shaped ceramic heat element 382 having a central opening 384 and an outer edge 386. Preferably, the heat element 382 is sized and configured to fit within the wipe housing 400 and immediately distal of the foam member 362 so that its opening 384 aligns with the foam member opening 364 and its edge 386 aligns generally with the foam member outer wall 366. Preferably, the wipe layer 370 also encases the heat element 382.

A controller 390 preferably comprises a thermostat including one or more sensors and associated control circuitry to both sense the temperature of fluids entrained within the foam member 362 and to regulate delivery of electricity to the heat element 382 in order to keep the entrained fluid within a desired temperature range. A first power wire 392 delivers power to the controller 390 and a second power wire 394 connects a power source to the heat element 382 at the edge 386 of the heat element 382. A control wire 396 extends from the controller 392 to the edge 386 of the heat element 382. The first and second power wires 392, 394 can extend proximally and terminate at wire ends 398 that can selectively be attached to a power source, as will be discussed in more detail below.

Continuing with reference to FIGS. 20-26, the wipe housing 400 is generally tubular, extending from a proximal end 402 to a distal end 404. A controller slot 406 formed within the wipe housing 400 preferably is sized and configured so that the controller 390 and portions of the first power wire 392 and control wire 396 fit therein. Preferably, the controller 390 is in contact with the wipe assembly 360 so that a sensor can measure the temperature of entrained fluid. Elongated wire races 408 are formed in an outer wall of the wipe housing 400 and extending from a proximal end toward the distal end. The races 408 are configured to receive the second power wire 394 and control wire 396 therewithin. The second power wire 394 and control wire 396 extend along the races 408 toward the distal end 404 of the housing 400 until they are aligned with the edge 386 of the heat element 382, at which point they change course to connect and provide power to the heat element 382.

The first power wire 392 and second power wire 394 preferably extend proximally beyond the proximal end 402 of the wipe housing 400. In the illustrated embodiment, however, each of these wires 392, 394 has a jog 409 immediately distal of the proximal end 402 at which the wire extends radially outwardly before again extending proximally toward the wire ends 398. In this manner, controller 390 and associated wires 392, 394, 396 effectively deliver regulated power to the heat element 382 while not interfering with any other structure that may extend into the wipe housing 400.

An elongated gas inlet slot 410 extends distally from the proximal end 402 of the wipe housing 400. Preferably, the gas inlet slot 410 is sized and configured so that when the wipe cartridge assembly 340 is inserted into the trocar housing 302, the gas inlet slot 410 is aligned with the gas inlet 350 (see FIG. 20), enabling unrestricted flow of insufflation gas into the trocar housing 302. It is to be understood that, in other embodiments, rather than an elongated slot, an aperture could be provided for alignment with the gas inlet 350. As noted above, the gas inlet 350 can also be used to deliver cleaning fluid to the wipe assembly 360. For example, as shown in FIG. 20, the gas inlet 350 opens into the wipe housing 400 immediately above and partially overlapping the wipe assembly 360. As such, prior to a medical procedure, an elongated tool such as a syringe or pipette can be advanced through the gas inlet 350 into engagement with or immediately adjacent the wipe assembly 360 so as to inject cleaning fluid into or onto the wipe assembly 360, which cleaning fluid will become entrained within the foam member 362. Preferably, the gas inlet 350 is positioned relative to the wipe assembly so that a straight access path is defined through the gas inlet to a point at or adjacent the wipe assembly in order to facilitate such fluid delivery.

A plurality of posts 414 extend distally from the distal end 404 of the wipe housing 400. A plurality of clips 416 are also incorporated into the wipe housing 400, preferably being defined between elongated clip slots 418. Preferably each clip 416 extends distally beyond the distal end 404 and terminates in an inwardly-directed projection 419.

With continued reference to FIGS. 20-26, the wipe assembly 360 and heat element 382 are sandwiched between the wipe housing 400 and a housing receiver 420. The housing receiver 420 preferably comprises a transverse wall 422 having a central opening 424 that aligns with a tubular distal portion 426 that extends distally from a distal surface of the transverse wall 422. An outer edge of the transverse wall 422 preferably has a diameter that is generally the same as the outer diameter of the wipe housing 400. A plurality of clip receivers 430 are formed in the transverse wall 422 and configured to generally align with clips 416 so that as the wipe housing 400 is advanced into contact with the housing receiver 420, the projections 419 of the clips 416 engage the distal surface of the transverse wall 422, preferably holding the wipe housing 400 in tight engagement with the housing receiver 420. In the illustrated embodiment, the cutouts 379 of the wipe layer 370 are also aligned with the clip receivers 430 when the wipe housing 400 is advanced into engagement with the housing receiver 420. As such, the clips 416 can easily pass by the wipe layer 370 through the cutouts 379 and into engagement with the clip receivers 430.

A plurality of receiver slots 432 are also formed in the edge 428 and configured to receive respective ones of the posts 414. Preferably, each post is aligned with one of the slots 376 of the wipe layer 370 and extends therethrough. Each post 414 can deform the associated slot 376 into an aperture 378 adjacent the wipe layer edge 374 as the post 414 passes therethrough. The posts 414 extend through the apertures 378 and into the receiver slots 432, so that the wipe layer 370 is held securely in place with its edge 374 sandwiched between the distal end 404 and the transverse wall 422. The wipe layer 370 encases the foam member 362 and heat element 382. As such, the wipe assembly 360 and heat element 382 are securely held in place within the wipe housing 400. Notably, the second power wire 394 and control wire 396 can gain access to be connected to the heat element 382 by extending through the clip slots 418 that are aligned with the races 308.

In the illustrated embodiment, the posts 414 are spaced radially inwardly from the outer surface of the wipe housing 400, and even partially radially inwardly from the inner surface of the wipe housing 400. Each illustrated post 414 includes a proximally-extending post base 434 formed as part of the inner surface of the wipe housing 400. In the illustrated embodiment, elongated thinned portions 436 of the wipe housing 400 are aligned with clips 416, enhancing the ability of the clips 416 to operate and to fit appropriately within the trocar housing 302.

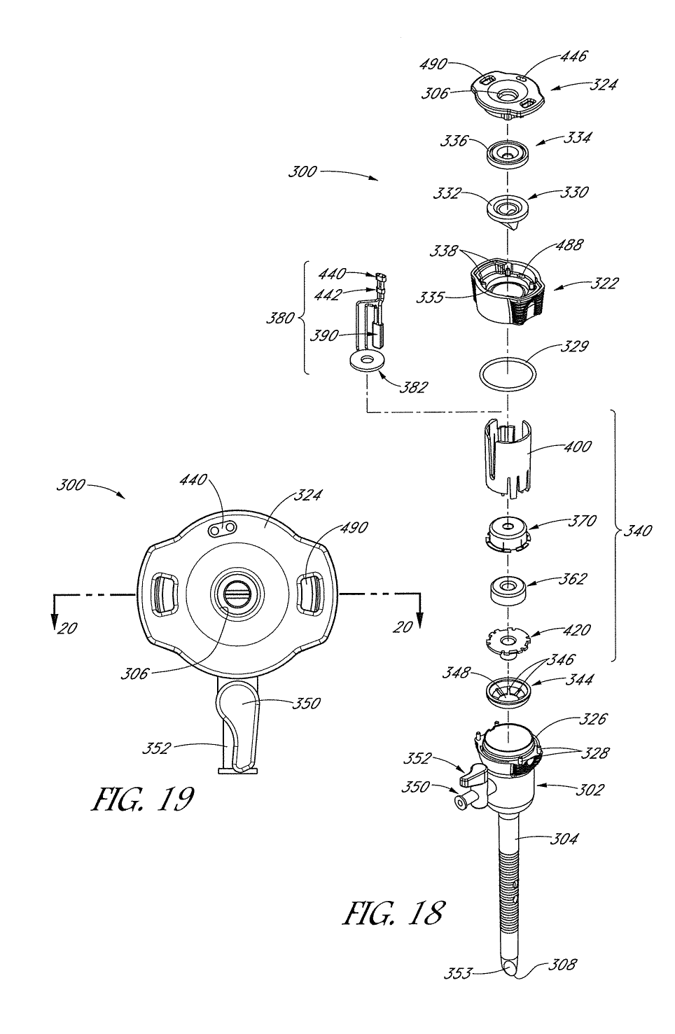

With reference again to FIGS. 18 and 19, preferably the wire ends 398 of the heater assembly 380 are supported by a contact block 440, which preferably is formed of an electrically insulative material. A plug 442 can also be provided to support portions of the first and second power wires 392, 394. A block aperture 446 is formed in the cap 324, and receives the contact block 440 therein so that the wire ends 398 are accessible for electrical connection. In the illustrated embodiment, a plug aperture 448 is formed in the seal housing 322 at a position radially outwardly from the seal housing seat 335 and is configured to support the plug 442. As such, a passage is provided through the seal housing 322 and cap 324 through which the power wires 392, 394 extend. As shown, the passage is formed radially outwardly from, for example, the seal 334 and the duckbill valve 330 so the wires 392, 394 do not interfere therewith.

With reference next to FIGS. 16-19 and 27-28, the obturator handle 316 includes an insert 450 enclosed between a base 452 and a cover 454. The insert 450 preferably comprises a compressible body 456 that supports opposing buttons 460 and associated clips 462. Insert supports 464 extend proximally from the base 452 to support the body 456. The clips 462 extend through clip slots 466 formed in the base 452, and the buttons 460 extend through button slots 468 formed in the cover 454. A battery 470 is supported on the base 452. An anode strip 472 is connected to an anode of the battery 470 and is supported within an anode race 474 defined in the base 452 and leading to one of a pair of power apertures 476. A pair of conductive, spring-biased plungers 480 are received in the pair of power apertures 476. The anode strip 472 is connected to one of the plungers 480. A cathode strip 478 is connected to a cathode of the battery 470 and extends to the other of the plungers 480. The spring-biased plungers 480 depend distally from the base 452 and are aligned with the wire ends 398 in the contact block 446 so that when the obturator 301 is engaged with the trocar 300 as depicted in FIG. 17, electric power is communicated from the battery 470 to the heater assembly 380.

With reference next to FIGS. 16-20, a pair of receiver apertures 490 are formed through the cap 324 so as to be aligned with the clips 462 of the obturator 301. A latch receiver 492 is formed at each receiver aperture 490. When the obturator 301 is engaged with the trocar 300 as depicted in FIG. 17, the clips 462 extend through the receiver apertures 490 and engage the latch receivers 492 so that the obturator 301 is held securely onto the trocar 300 with the plungers 480 engaged with the wire ends 398. As such, in the illustrated embodiment, engaging the obturator 301 with the trocar 300 initiates provision of power from the battery 370 to the heater assembly 380. In a preferred embodiment, the obturator 301 is engaged with the trocar 300 during preparation for a procedure. As such, the fluid entrained in the wipe assembly 360 is appropriately heated. Once access to the patient's abdomen has been accomplished using the engaged obturator 301/trocar 300, the obturator 301 can be removed and the trocar 300 is properly heated and ready to receive a laparoscope. Depressing the obturator buttons 468 will disengage the clips 462 from the latch receivers 492 so that the obturator 301 can be removed from the trocar 300.

With specific reference next to FIGS. 18 and 20, preferably the seal housing 322 comprises a distally-extending extension 498 that terminates in an extension opening 500. In the illustrated embodiment an inner surface of the access opening 306 functions as the proximal fulcrum surface 306 and an inner surface of the extension opening 500 functions as the distal fulcrum surface 500. The laparoscope 50 can be selectively engaged with the proximal and distal fulcrum surfaces 306, 500 in an off-axis configuration to wipe the laparoscope lens 180 and/or to apply heated fluid to the lens 180 using wiping actions such as the back-and-forth, twist, and swirl actions described above.

In the illustrated embodiment, a battery portion 480 of the obturator cover 454 is sized and configured to accommodate the battery 470. In additional embodiments, the battery portion could be included in the trocar housing and an actuator button provided to selectively connect the battery electrically with the heater assembly. As such, the heater assembly can remain energized throughout a procedure.

The present embodiment is disclosed and configured in connection with cleaning the distal end and lens of a laparoscope. However, it is to be understood that the principles and structures of the embodiments discussed herein can be used to warm and clean other medical scopes that may use other types and configurations of surgical access devices.

The embodiments discussed above have disclosed structures with substantial specificity. This has provided a good context for disclosing and discussing inventive subject matter. However, it is to be understood that other embodiments may employ different specific structural shapes and interactions.

Although inventive subject matter has been disclosed in the context of certain preferred or illustrated embodiments and examples, it will be understood by those skilled in the art that the inventive subject matter extends beyond the specifically disclosed embodiments to other alternative embodiments and/or uses of the invention and obvious modifications and equivalents thereof. In addition, while a number of variations of the disclosed embodiments have been shown and described in detail, other modifications, which are within the scope of the inventive subject matter, will be readily apparent to those of skill in the art based upon this disclosure. It is also contemplated that various combinations or subcombinations of the specific features and aspects of the disclosed embodiments may be made and still fall within the scope of the inventive subject matter. Accordingly, it should be understood that various features and aspects of the disclosed embodiments can be combined with or substituted for one another in order to form varying modes of the disclosed inventive subject matter. Thus, it is intended that the scope of the inventive subject matter herein disclosed should not be limited by the particular disclosed embodiments described above, but should be determined only by a fair reading of the claims that follow.

1. A method of cleaning a medical scope lens, comprising:
moving a medical scope longitudinally along a passage defined within and extending through a cannula and a head portion of a surgical access device, a head portion space defined in the head portion between the passage and an outer wall of the head portion, a wipe surface of a wipe assembly disposed within the head portion space;
moving the medical scope along the passage so that a lens of the medical scope is positioned within the passage and proximal of the wipe surface;
engaging the medical scope with a proximal fulcrum surface of the surgical access device;
rotating the medical scope about a rotation axis that is transverse to an axis of the passage to an off-axis orientation in which an axis of the medical scope crosses the axis of the passage and the lens is within the head portion space, wherein rotating the medical scope to the off-axis orientation comprises rotating the medical scope about the proximal fulcrum surface;
rotating the medical scope about the proximal fulcrum surface until the medical scope engages a distal fulcrum surface of the surgical access device, the distal fulcrum surface being proximal of the wipe surface;
moving the medical scope so that the lens is engaged with the wipe surface while the medical scope axis is in the off-axis configuration; and
moving the medical scope so that the lens is wiped across the wipe surface.

2. (canceled)

3. (canceled)

4. The method of claim 1, additionally comprising moving the medical scope lens back into the passage after the lens is wiped across the wipe surface, and advancing the medical scope along the passage through a distal opening of the cannula and into a surgical field.

5. The method of claim 1, additionally comprising rotating the medical scope about the proximal fulcrum surface and out of engagement with the distal fulcrum surface so as to wipe the lens across the wipe surface along a back-and-forth wipe path.

6. The method of claim 1, additionally comprising rotating the medical scope about the medical scope axis while the medical scope is engaged with both of the proximal and distal fulcrum surfaces and the wipe surface so as to wipe the lens on the wipe surface along a twisting wipe path.

7. The method of claim 1, wherein the proximal and distal fulcrum surfaces are circumferential, and additionally comprising sliding the medical scope about the circumference of both the proximal and distal fulcrum surfaces so as to wipe the lens on the wipe surface along a swirl flow path.

8. The method of claim 1, additionally comprising moving the medical scope so that the lens of the medical scope is within a surgical field, then engaging a computer-controlled robot with the medical scope and initiating a cleaning cycle, wherein during the cleaning cycle the computer-controlled robot performs the steps of moving the medical scope along the passage so that the lens is positioned proximal of the wipe surface, rotating the medical scope to the off-axis orientation, moving the medical scope so that the lens is engaged with the wipe surface while the medical scope axis is in the off-axis configuration, and moving the medical scope so that the lens is wiped across the wipe surface.

9. The method of claim 1, additionally comprising identifying a visual cue within the surgical access device visible through the lens in order to determine that the lens is adjacent the wipe assembly.

10. The method of claim 1, wherein the wipe assembly comprises a foam member, and additionally comprising entraining a cleaning fluid in the foam member, and cleaning fluid is transferred from the foam member to the lens when the lens is wiped across the wiping surface.

11. The method of claim 10, additionally comprising warming the cleaning fluid.

12. The method of claim 10, additionally comprising advancing an elongated tube through an inlet formed through a side wall of the surgical access device so that an opening of the elongated tube is adjacent or engaged with the wipe assembly, and delivering cleaning fluid onto the wipe assembly, and further comprising delivering an insufflation gas from a source of insufflation gas through the inlet and into the surgical access device.

13.-20. (canceled)

21. The method of claim 1, wherein a head portion width is greater than a passage width, and the passage width is the same as a cannula width.

22. The method of claim 1, wherein the lens of the medical scope is spaced distally and radially outwardly from the distal fulcrum surface when the medical scope is engaged with both the proximal fulcrum surface and the distal fulcrum surface.

23. The method of claim 1, wherein the distal fulcrum surface is distal of a valve within the surgical access device.

24. The method of claim 23, wherein the proximal fulcrum surface is proximal of the valve.

25. A method of cleaning a medical scope lens, comprising:
moving a medical scope along a passage defined within a surgical access device so that a lens of the medical scope is drawn proximally through a cannula portion of the surgical access device into a head portion of the surgical access device, the passage being aligned with the cannula portion, the head portion being proximal of the cannula portion, distal of a valve of the surgical access device, and having a head portion width greater than a cannula portion width, a cleaning zone defined within the head portion between the passage and an outer wall of the cleaning portion;
moving the medical scope so that the lens is moved radially out of the passage and into the cleaning zone, comprising:
engaging the medical scope with a proximal fulcrum surface of the surgical access device; and
rotating the medical scope about a rotation axis that is at the proximal fulcrum surface and is transverse to an axis of the passage until the medical scope contacts a distal fulcrum surface that is proximal of a wipe surface disposed within the cleaning zone and radially spaced from the outer wall of the head portion;
moving the medical scope so that the lens is engaged with the wipe surface; and
moving the medical scope so that the lens is wiped across the wipe surface.

26. (canceled)

27. The method of claim 25, wherein moving the medical scope so that the lens is engaged with the wipe surface comprises advancing the medical scope while keeping the medical scope in contact with both the proximal fulcrum surface and the distal fulcrum surface.

28. The method of claim 27, additionally comprising engaging the medical scope with a computer-controlled robot and directing the computer-controlled robot to perform a cleaning cycle, wherein performing the cleaning cycle comprises moving the medical scope proximally along the passage, identifying a visual cue within the surgical access device indicating that the lens is proximal of the wipe surface, engaging the medical scope with the proximal fulcrum surface, rotating the medical scope about the rotation axis until the medical scope contacts the distal fulcrum surface, advancing the medical scope while keeping the medical scope in contact with both the proximal fulcrum surface and the distal fulcrum surface, determining that the lens is engaged with the wipe surface, and wiping the lens across the wipe surface.

29. The method of claim 27, wherein an axis of the medical scope intersects the wipe surface when the medical scope is in contact with both the proximal fulcrum surface and the distal fulcrum surface.

30. The method of claim 1, wherein an axis of the medical scope intersects the wipe surface when the medical scope is simultaneously in contact with both the proximal fulcrum surface and the distal fulcrum surface, and comprising advancing the medical scope toward the wipe surface while maintaining the medical scope simultaneously in contact with both the proximal fulcrum surface and the distal fulcrum surface.
